(19) United States
(12) Patent Application Publication
(10) Pub. No.: US 2012/0209316 A1
(43) Pub. Date: Aug. 16, 2012

(54) MULTIFUNCTIONAL GROOMING TWEEZERS

(75) Inventors: Jacquelyn Tarlton, Miami, FL (US); Dawn Sole, Miami, FL (US)

(73) Assignee: TASO, LLC, Miami, FL (US)

(21) Appl. No.: 13/027,179

(22) Filed: Feb. 14, 2011

(51) Int. Cl. A61B 17/50

(52) U.S. Cl. 606/210

(57) ABSTRACT

Multifunctional grooming tweezers can include a first prong extending from a proximal end to a distal end and having an inner surface on one side of the first prong and an outer surface on an opposite side of the first prong. The tweezers can further include a second prong extending from a proximal end to a distal end and having an inner surface on one side of the second prong and an outer surface on an opposite side of the second prong. The first and second prongs are fixedly attached to each other solely at their proximal ends such that the distal ends of the first and second prongs are free to move relative to each other. The inner surfaces of the first and second prongs face toward each other and are spaced apart from each other along substantially the entire length of the first and second prongs, and the outer surfaces of the first and second prongs face away from each other. A file portion on the first prong can have a file surface substantially co-planar with the outer surface of the first prong and configured to grind down and shape an edge of a nail. A buffer portion on the second prong can have a buffing surface substantially co-planar with the outer surface of the second prong and configured to polish the nail.

### BACKGROUND

1. Field of Use

Embodiments of the present invention relate generally to grooming tweezers, and more specifically to multifunctional grooming tweezers.

2. Description of the Related Art

Tweezers are commonly used for grooming, for example, eyebrows. They can be carried by individuals (e.g., in purses) for easy access and use, for example, when in the car, at the office or while traveling. Other grooming devices are sometimes also carried by individuals, for example in purses, for use while away from the home. One such device is a nail file. However, a purse can become cluttered with such multiple grooming devices, making it more time-consuming and difficult for a person to readily find and access them when in a hurry. Additionally, where the purse is small (e.g., a clutch-purse) and has a limited amount of space, carrying multiple individual grooming devices, along with other essentials (e.g., ID, money, keys, mobile phone), is difficult, if not impossible. Therefore, there is a need for compact multi-functional tweezers that can be used to groom additional parts of the body, such as nails.

### SUMMARY

In accordance with one embodiment, multifunctional grooming tweezers are provided. The tweezers comprise a first prong extending from a proximal end to a distal end and having an inner surface on one side of the first prong and an outer surface on an opposite side of the first prong. The tweezers further comprise a second prong extending from a proximal end to a distal end and having an inner surface on one side of the second prong and an outer surface on an opposite side of the second prong. The first and second prongs are fixedly attached to each other solely at their proximal ends such that the distal ends of the first and second prongs are free to move relative to each other. The inner surfaces of the first and second prongs face toward each other and are spaced apart from each other along substantially the entire length of the first and second prongs, and the outer surfaces of the first and second prongs face away from each other. The tweezers further comprise a file portion on the first prong and having a file surface substantially co-planar with the outer surface of the first prong, the file surface configured to grind down and shape an edge of a nail, and a buffer portion on the second prong and having a buffing surface substantially co-planar with the outer surface of the second prong, the buffing surface configured to smooth the nail.

In accordance with another embodiment, multifunctional grooming tweezers are provided. The tweezers comprise a first prong extending from a proximal end to a distal end and having an inner surface on one side of the first prong and an outer surface on an opposite side of the first prong. The tweezers further comprise a second prong extending from a proximal end to a distal end and having an inner surface on one side of the second prong and an outer surface on an opposite side of the second prong. The first and second prongs are fixedly attached to each other solely at their proximal ends such that the distal ends of the first and second prongs are free to move relative to each other. The inner surfaces of the first and second prongs face toward each other and are spaced apart from each other along substantially the entire length of the first and second prongs, and the outer surfaces of the first and second prongs face away from each other. The tweezers further comprise a file portion removably attached to the first prong and having a file surface generally co-planar with the outer surface of the first prong, the file surface configured to grind down and shape an edge of a nail, and a buffer portion removably attached to the second prong and having a buffing surface generally co-planar with the outer surface of the second prong, the buffing surface configured to smooth the nail.

In accordance with still another embodiment, a multifunctional grooming tweezers kit is provided. The kit comprises a pair of tweezer prongs comprising a first prong extending from a proximal end to a distal end and having an inner surface on one side of the first prong and an outer surface on an opposite side of the first prong, and a second prong extending from a proximal end to a distal end and having an inner surface on one side of the second prong and an outer surface on an opposite side of the second prong. The first and second prongs are fixedly attached to each other solely at their proximal ends such that the distal ends of the first and second prongs are free to move relative to each other. The inner surfaces of the first and second prongs face toward each other and are spaced apart from each other along substantially the entire length of the first and second prongs, and the outer surfaces of the first and second prongs face away from each other. The kit further comprises a file member removably coupleable to the first prong so that a file surface thereof is generally co-planar with the outer surface of the first prong, the file surface configured to grind down and shape an edge of a nail. Additionally, the kit comprises a buffer member removably coupleable to the second prong so that a buffing surface thereof is generally co-planar with the outer surface of the second prong, the buffing surface configured to smooth the nail.

In accordance with yet another embodiment, a kit for use with multifunctional tweezers is provided. The kit comprises one or more elongate members comprising a first surface on one side of the one or more elongate members chosen from a group consisting of a file surface and a buffing surface, the one or more elongate members configured to be attached to one of a pair of tweezer prongs such that said first surface is disposed on an outer surface of the prong. The one or more elongate members further comprise a second surface on an opposite side of the elongate members configured to contact a surface of the prong when the elongate member is attached to the prong. The one or more elongate members have a length between a proximal end and a distal end thereof that is shorter than the length of the tweezer prong.

### BRIEF DESCRIPTION OF THE DRAWINGS

FIG. 1 is a top perspective schematic view of one embodiment of multi-functional tweezers.

FIG. 2 is a top planar schematic view of the multi-functional tweezers in

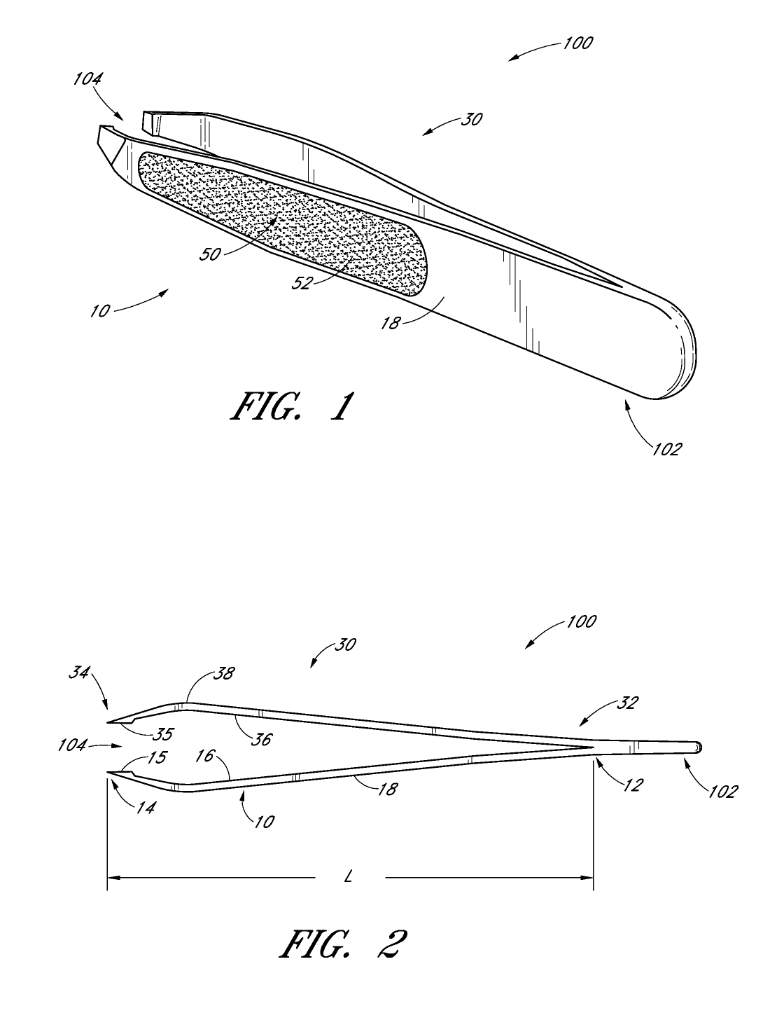

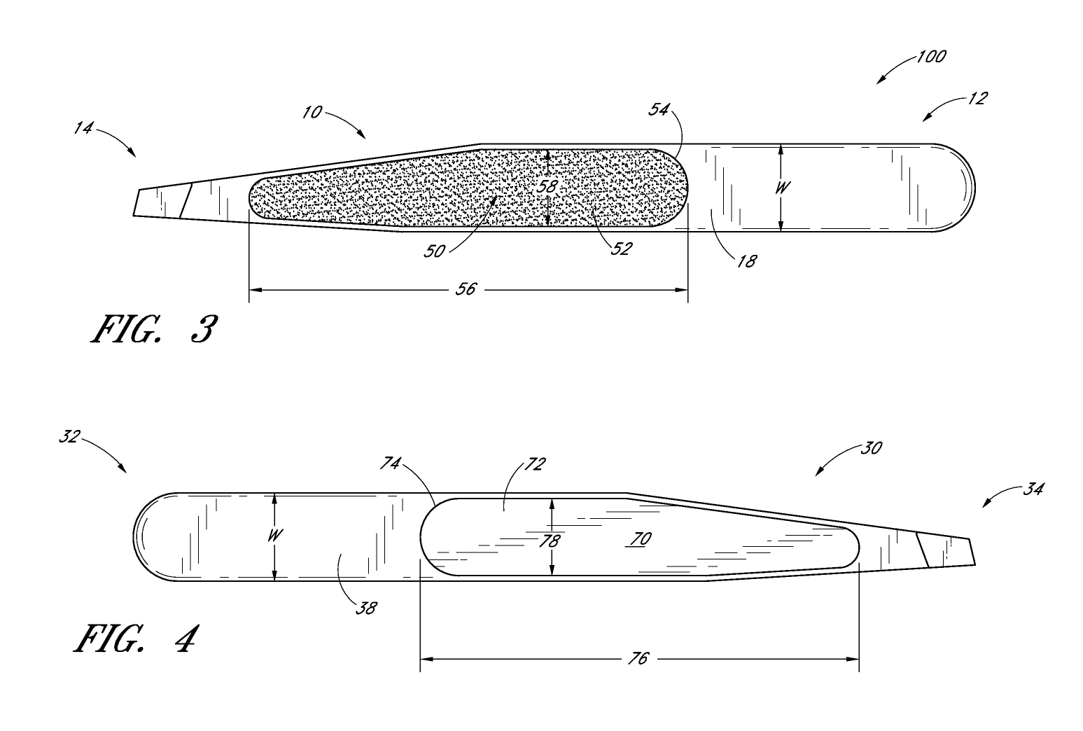

FIG. 1.

FIG. 3 is left-side schematic view of the multi-functional tweezers in FIG. 1.

FIG. 4 is a right-side schematic view of the multi-functional tweezers in FIG. 1.

FIG. 5 is a perspective exploded view of the multi-functional tweezers in FIG. 1.

FIG. 6 is a top planar exploded view of the multi-functional tweezers in FIG. 1.

### DETAILED DESCRIPTION

In the following detailed description, terms of orientation such as “top,” “bottom,” “upper,” “lower,” “front,” “inner,” “outer,” “rear,” and “end” are used herein to simplify the description of the context of the illustrated embodiments. Likewise, terms of sequence, such as “first” and “second,” are used to simplify the description of the illustrated embodiments. Because other orientations and sequences are possible, however, the present invention should not be limited to the illustrated orientation. Those skilled in the art will appreciate that other orientations of the various components described above are possible. Additionally, as used herein, the term “tweezer” and “tweezers” are used interchangeably and are both meant to refer to a single device with two prongs cantilevered relative to each other at their distal ends, such that the prongs can move relative to each other.

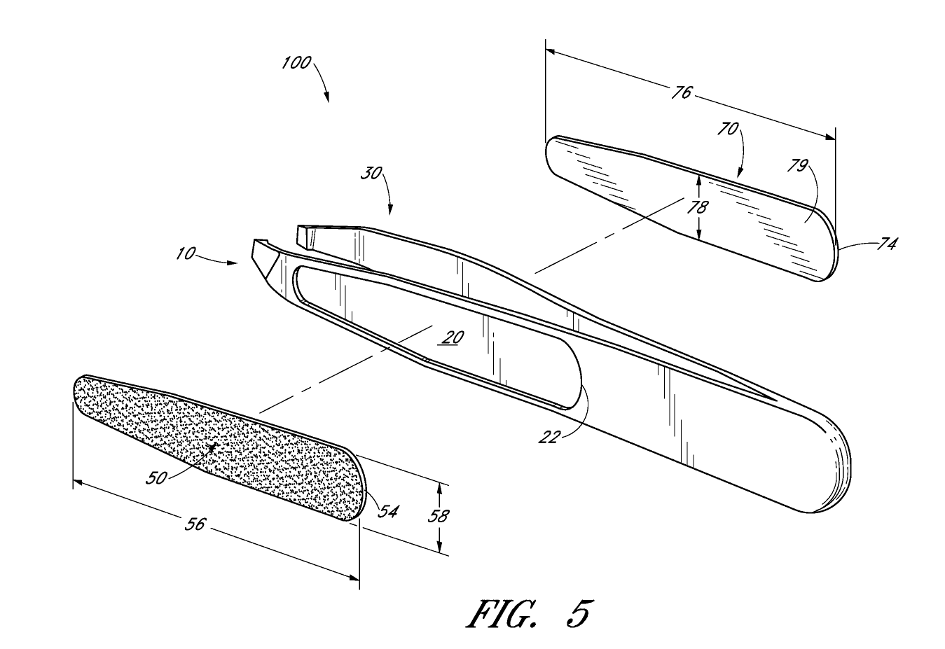

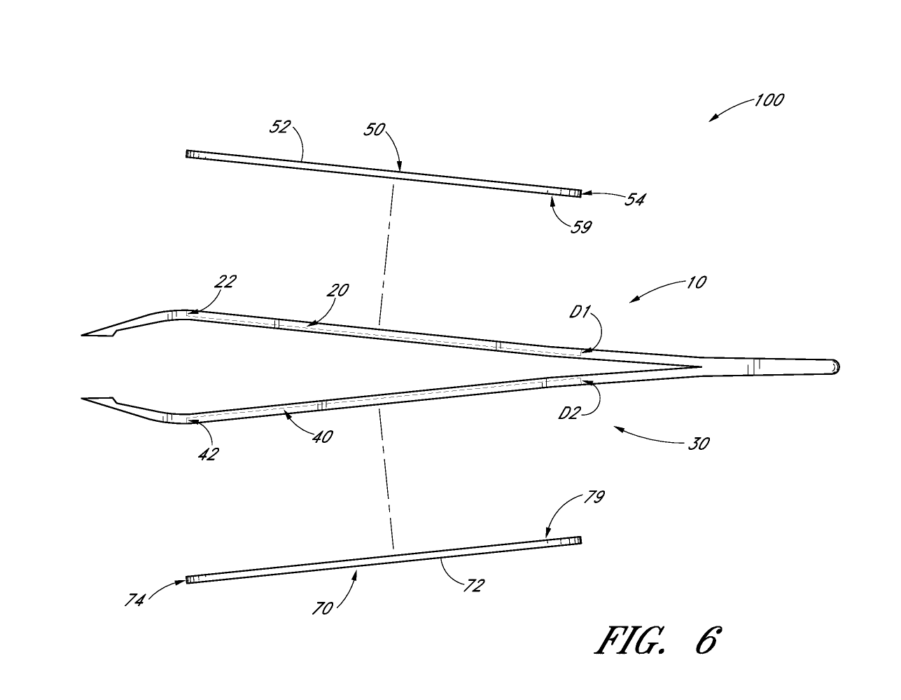

FIGS. 1 and 2 shows one embodiment of a multifunctional set of tweezers 100 with a first prong 10 and a second prong 30, each extending along a length L and having a width W. In some embodiments, the length L can coincide with the length of the tweezers 100. In other embodiments, the length L can be shorter than the length of the tweezers 100. The first prong 10 can extend from a proximal end 12 to a distal end 14, and have an inner surface 16 on one side of the prong 10 and an outer surface 18 on an opposite side of the prong 10. Similarly, the second prong 30 can extend from a proximal end 32 to a distal end 34, and have an inner surface 36 on one side of the prong 30 and an outer surface 38 on an opposite side of the prong 30. In one embodiment, the inner surfaces 16, 36 and outer surfaces 18, 38 of the prongs can be generally planar, such that the prongs 10, 30 can each have a generally rectangular transverse cross-section. In another embodiment, at least a portion of one or more of the inner surfaces 16, 36 and outer surfaces 18, 38 can be non-planar (e.g., curved). In the illustrated embodiment, the inner surfaces 16, 36 of the prongs 10, 30 are oriented such that they are opposite and face toward each other along substantially the entire length L of the prongs 10, 30, while the outer surfaces 18, 38 are oriented such that they face away from each other.

With continued reference to FIGS. 1 and 2, the prongs 10, 30 can be fixedly attached at their proximal ends 12, 32. In one embodiment, the proximal ends 12, 32 can be fixedly attached to each other with an adhesive (e.g., glue). In another embodiment, the proximal ends 12, 32 can be fixedly attached to each other with one or more fasteners (e.g., with threaded fasteners, pins or rivets). In still another embodiment, the proximal ends 12, 32 can be fixedly attached to each other via a monolithic proximal end 102 of the tweezers 100. In one embodiment, the proximal ends 12, 32 of the prongs 10, 30 can be aligned with the proximal end 102 of the tweezers 100. In another embodiment, the proximal ends 12, 32 of the prongs 10, 30 can be positioned distally of the proximal end 102 of the tweezers 100. As shown in FIG. 2, the tweezers 100 can have gripping portions 15, 35 on the prongs 10, 30 at the distal end 104 that can be used to grip a body part (e.g., eyebrow hair).

As best shown in FIGS. 1 and 3, the first prong 10 can include a file portion 50 with a file surface 52. The file surface 52 can be defined by a circumference 54 and have a length 56 and width 58. In one embodiment, the length 56 of the file portion 50 can be shorter than the length L of the prong 10. For example, in one embodiment the length 56 of the file portion 50 can be between about 20% and about 80% of the length L of the prong 10. In still another embodiment, the length 56 of the file portion 50 can be about 50% or less of the length L of the prong 10. However, the file portion 50 can have other suitable lengths. In one embodiment, the length 56 of the file portion 50 can be generally equal to the length L of the prong 10. In the illustrate embodiment, the width 58 of the file portion 50 is generally equal to the width W of the prong 10. In another embodiment, the width 58 of the file portion 50 can be identical to the width W of the prong 10.

In one embodiment, the file surface 52 can include an abrasive surface. For example, in one embodiment, the file surface 52 can include an abrasive surface with a plurality of particles of an abrasive material (e.g., emery). In another embodiment, the file surface 52 can include an abrasive surface with a plurality of ridges (e.g., parallel ridges). However, the file surface 52 can have an abrasive surface with other suitable configurations.

As best shown in FIG. 4, the second prong 30 can include a buffer portion 70 with a buffing surface 72 that can, for example, smooth, shape and/or polish a nail. The buffing surface 72 can be defined by a circumference 74 and have a length 76 and width 78. In one embodiment, the length 76 of the buffer portion 70 can be shorter than the length L of the prong 30. For example, in one embodiment the length 76 of the buffer portion 70 can be between about 20% and about 80% of the length L of the prong 30. In still another embodiment, the length 76 of the buffer portion 70 can be about 50% or less of the length L of the prong 30. However, the buffer portion 70 can have other suitable lengths. In one embodiment, the length 76 of the buffer portion 70 can be generally equal to the length L of the prong 30. In the illustrated embodiment, the width 78 of the buffer portion 70 is generally equal to the width W of the prong 30. In another embodiment, the width 78 of the buffer portion 70 can be identical to the width W of the prong 30.

In one embodiment, the buffing surface 72 can be a substantially smooth surface. In another embodiment, the buffing surface 72 can also be a generally soft or resilient surface (e.g., padded, sponge-like). However, the buffing surface 72 can have other suitable configurations. In one embodiment, the file and buffer portions 50, 70 can be integral with the prongs 10, 30 so that they form part of one monolithic tweezers unit 100.

FIGS. 5 and 6 show one embodiment of the tweezers 100, where the file portion 50 and buffer portion 70 are separate elongate members that can be attached to the prongs 10, 30. In one embodiment, the file and buffer portions 50, 70 can be removably attached to the prongs 10, 30. In another embodiment, the file and buffer portions 50, 70 can be permanently attached to the prongs 10, 30.

As illustrated in FIGS. 5 and 6, the first prong 10 can have a recess 20 on its outer surface 18 that receives at least a portion of the file portion 50. In one embodiment, the recess 20 can have a circumference 22 that generally corresponds to the circumference 54 of the file portion 50. In another embodiment, the circumference 22 can be slightly smaller than the circumference 54 of the file portion 50, such that the file portion 50 can be press-fit into the recess 20. The recess 20 has a depth D1 such that the file surface 52 is generally co-planar with the outer surface 18 of the first prong 10 when the file portion 50 is attached to the first prong 10. For example, in one embodiment, the file surface 52 can be aligned with the outer surface 18, such that the surfaces 52, 18 extend along the same plane. In another embodiment, the file surface 52 can be spaced above the outer surface 18, such that the surfaces 52, 18 extend along generally parallel planes and the file surface is raised relative to the outer surface 18. In still another embodiment, the file surface 52 can be spaced below the outer surface 18, such that the surfaces 52, 18 extend along generally parallel planes and the file surface 52 is recessed relative to the outer surface 18.

With continued reference to FIGS. 5 and 6, the second prong 30 can have a recess 40 on its outer surface 38 that receives at least a portion of the buffer portion 70. In one embodiment, the recess 40 can have a circumference 42 that generally corresponds to the circumference 74 of the buffer portion 70. In another embodiment, the circumference 42 can be slightly smaller than the circumference 74 of the buffer portion 70, such that the buffer portion 70 can be press-fit into the recess 40. The recess 40 has a depth D2 such that the buffing surface 72 is generally co-planar with the outer surface 38 of the second prong 30 when the buffer portion 70 is attached to the second prong 30. For example, in one embodiment, the buffing surface 72 can be aligned with the outer surface 38, such that the surfaces 72, 38 extend along the same plane. In another embodiment, the buffing surface 72 can be spaced above the outer surface 38, such that the surfaces 72, 38 extend along generally parallel planes and the buffing surface 72 is raised relative to the outer surface 38. In still another embodiment, the buffing surface 72 can be spaced below the outer surface 38, such that the surfaces 72, 38 extend along generally parallel planes and the buffing surface 72 is recessed relative to the outer surface 38.

As discussed above, in one embodiment the file and buffer portions 50, 70 can be removably attached to the prongs 10, 30 via a press-fit connection. In another embodiment, the file and buffer portions 50, 70 can have fastening mechanisms for removably coupling to the prongs 10, 30. For example, the file and buffer portions 50, 70 can have a hook or loop fastener on a bottom surface thereof for coupling to a corresponding loop or hook fastener on the prongs 10, 30 (e.g., on the outer surfacel8, 38 of the prongs 10, 30). In another embodiment, the file and buffer portions 50, 70 can have a snap fastener to snap to a corresponding receiving member or aperture on the prongs 10, 30. In still another embodiment, the file and buffer portions 50, 70 can have clamps that clamp to the prongs 10, 30 (e.g., clamp about the edges of the prongs) to removably fasten the file and buffer portions 50, 70 to the prongs 10, 30. In yet another embodiment, the file and buffer portions 50, 70 can be fastened to the prongs 10, 30 via an adhesive on a bottom surface 59, 79 of the file and buffer portions 50, 70. For example, the file and buffer portions 50, 70 can have an adhesive on their respective bottom surfaces 59, 79 that is covered by a removable outer layer (not shown) when the file and buffer portions 50, 70 are separate from the tweezers 100. When a user wants to attach the file and buffer portions 50, 70 to the prongs 10, 30, the user can remove the outer layer from the bottom surfaces 59, 79 of the file and buffer portions 50, 70 and attach the file and buffer portions 50, 70 to the prongs 10, 30 (e.g., attach them to the recesses 20, 40 of the prongs 10, 30).

The file and/or buffer portions 50, 70 can be packaged as part of a kit, where the kit can optionally include a multifunctional tweezer 100. In another embodiment, the kit can include only one or more of the file and buffer portions 50, 70, but not include the multifunctional tweezers 100.

Advantageously, a user can easily replace the file or buffer portions 50, 70 of the multifunctional tweezer 100 when they wear out, without having to purchase a whole new tweezer 100. Therefore, the multifunctional tweezer 100 diminishes waste by allowing the user to replace the file and buffer portions 50, 70 while keeping the same tweezer 100. Additionally, the multifunctional tweezer 100 allows the user to reduce the number of beauty aids she carries in a purse because a separate file and buffer need not be carried. Moreover, by reducing the number of beauty aids the user has to carry, the multifunctional tweezer 100 diminishes the amount of clutter in the user's purse. Additionally, because of its size, the multifunctional tweezer 100 can easily be carried in purses of varying size (e.g., in clutch purses), or in a pocket, allowing its use while outside the home.

The tweezers 100 can be made of any suitable material, such as metal or plastic, or other resilient material. As discussed above, the file portion 50 preferably includes an abrasive surface, which can include an abrasive material thereon. Additionally, the buffer portion 70 can have a resilient or padded material.

Of course, the foregoing description is that of certain features, aspects and advantages of the present invention, to which various changes and modifications can be made without departing from the spirit and scope of the present invention. Moreover, the multifunctional tweezers need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed multifunctional tweezers.

What is claimed is:

1. A multi-functional grooming tweezers, comprising:
a first prong extending from a proximal end to a distal end and having an inner surface on one side of the first prong and an outer surface on an opposite side of the first prong;
a second prong extending from a proximal end to a distal end and having an inner surface on one side of the second prong and an outer surface on an opposite side of the second prong, the first and second prongs fixedly attached to each other solely at their proximal ends such that the distal ends of the first and second prongs are free to move relative to each other, the inner surfaces of the first and second prongs facing toward each other and spaced apart from each other along substantially the entire length of the first and second prongs, the outer surfaces of the first and second prongs facing away from each other;
a file portion on the first prong and having a file surface generally co-planar with the outer surface of the first prong, the file surface configured to grind down and shape an edge of a nail; and
a buffer portion on the second prong and having a buffing surface generally co-planar with the outer surface of the second prong, the buffing surface configured to smooth the nail.

2. The tweezers of claim 1, wherein the file surface comprises an abrasive surface comprising a plurality of abrasive particles.

3. The tweezers of claim 1, wherein one or both of the file portion and buffer portion are removably attached to the first and second prongs.

4. The tweezers of claim 3, wherein one or both of the file portion and buffer portion are at least partially disposed in recesses on the outer surfaces of the first and second prongs.

5. The tweezers of claim 4, wherein one or both of the file and buffer portions are removably attached to the first and second prongs via a press-fit connection.

6. The tweezers of claim 1, wherein the file and buffer portions each have a length that is shorter than the length of the first and second prongs, respectively.

7. The tweezers of claim 1, wherein the file surface and outer surface of the first prong extend along a single plane, and wherein the buffing surface and the outer surface of the second prong extend along a single plane.

8. A multi-functional grooming tweezers, comprising:
a first prong extending from a proximal end to a distal end and having an inner surface on one side of the first prong and an outer surface on an opposite side of the first prong;
a second prong extending from a proximal end to a distal end and having an inner surface on one side of the second prong and an outer surface on an opposite side of the second prong, the first and second prongs fixedly attached to each other solely at their proximal ends such that the distal ends of the first and second prongs are free to move relative to each other, the inner surfaces of the first and second prongs facing toward each other and spaced apart from each other along substantially the entire length of the first and second prongs, the outer surfaces of the first and second prongs facing away from each other;
a file portion removably attached to the first prong and having a file surface generally co-planar with the outer surface of the first prong, the file surface configured to grind down and shape an edge of a nail; and
a buffer portion removably attached to the second prong and having a buffing surface generally co-planar with the outer surface of the second prong, the buffing surface configured to smooth the nail.

9. The tweezers of claim 8, wherein the file surface and outer surface of the first prong extend along a single plane, and wherein the buffing surface and the outer surface of the second prong extend along a single plane.

10. The tweezers of claim 8, wherein the file surface comprises an abrasive surface comprising a plurality of abrasive particles.

11. The tweezers of claim 8, wherein the file and buffer portions are at least partially disposed in recesses on the outer surfaces of the first and second prongs.

12. The tweezers of claim 8, wherein the file and buffer portions each have a length that is shorter than the length of the first and second prongs, respectively.

13. A multi-functional grooming tweezers kit, comprising:
a pair of tweezer prongs comprising:
a first prong extending from a proximal end to a distal end and having an inner surface on one side of the first prong and an outer surface on an opposite side of the first prong, and
a second prong extending from a proximal end to a distal end and having an inner surface on one side of the second prong and an outer surface on an opposite side of the second prong, the first and second prongs fixedly attached to each other solely at their proximal ends such that the distal ends of the first and second prongs are free to move relative to each other, the inner surfaces of the first and second prongs facing toward each other and spaced apart from each other along substantially the entire length of the first and second prongs, the outer surfaces of the first and second prongs facing away from each other;
a file member coupleable to the first prong so that a file surface thereof is generally co-planar with the outer surface of the first prong, the file surface configured to grind down and shape an edge of a nail; and
a buffer member coupleable to the second prong so that a buffing surface thereof is generally co-planar with the outer surface of the second prong, the buffing surface configured to smooth the nail.

14. The kit of claim 13, wherein the file surface comprises an abrasive surface comprising a plurality of abrasive particles.

15. The kit of claim 13, wherein the outer surfaces of the first and second prongs each have a recessed portion, the recessed portions configured to receive at least a portion of the file and buffer members.

16. The kit of claim 13, wherein the file and buffer members are removably coupleable to the first and second prongs, each of the file and buffer member having a length from a proximal end to a distal end thereof that is shorter than the length of the first and second prongs to which the file and buffer members are attached.

17. A kit for use with multifunctional tweezers, comprising:
one or more elongate members comprising a first surface on one side of the one or more elongate members chosen from a group consisting of a file surface and a buffing surface, the one or more elongate members configured to be attached to one of a pair of tweezer prongs such that said first surface is disposed on an outer surface of the prong, the one or more elongate members comprising a second surface on an opposite side of the elongate members configured to contact a surface of the prong when the elongate member is attached to the prong, the one or more elongate members having a length between a proximal end and a distal end thereof that is shorter than the length of the tweezer prong.

18. The kit of claim 17, wherein the one or more elongate members comprises a first elongate member having a file surface and a second elongate member having a buffing surface, the first and second elongate members coupleable to a separate prong of the tweezers such that the file and buffing surfaces are generally co-planar with an outer surface of the first and second prongs.

19. The kit of claim 17, wherein the one or more elongate members are at least partially insertable in recesses on an outer surface of the tweezer prongs, the one or more elongate members having a circumferential shape corresponding to a shape of the recesses on the tweezer prongs.

20. The kit of claim 19, wherein the one or more elongate members are removably attached to the tweezer prongs.

21. The kit of claim 19, wherein the one or more elongate members are permanently attached to the tweezer prongs.

22. The kit of claim 17, wherein the file surface comprises an abrasive surface comprising a plurality of abrasive particles.

23. The kit of claim 17, wherein the length of the one or more elongate members is between about 20% and about 80% of the length of the tweezer prongs.
